(19) United States
(12) Patent Application Publication
(10) Pub. No.: US 2016/0038079 A1
(43) Pub. Date: Feb. 11, 2016

(54) COMPUTER-READABLE RECORDING MEDIUM AND AROUSAL-LEVEL DETERMINING APPARATUS

(71) Applicant: FUJITSU LIMITED, Kawasaki-shi (JP)

(72) Inventors: Yasuhiko NAKANO, Kawasaki (JP); Satoshi SANO, Kawasaki (JP)

(73) Assignee: Fujitsu Limited, Kawasaki (JP)

(21) Appl. No.: 14/812,697

(22) Filed: Jul. 29, 2015

(30) Foreign Application Priority Data
Aug. 7, 2014 (JP) 2014-161920

(51) Int. Cl. A61B 5/00; A61B 5/18; A61B 5/024

(52) U.S. Cl. CPC A61B 5/4809; A61B 5/02405; A61B 5/18; A61B 5/7203; A61B 5/6893; A61B 5/7275; A61B 5/742

(57) ABSTRACT

An arousal-level determining apparatus according to a present embodiment determines a subject's arousal level on the basis of a biological signal detected from a subject. The arousal-level determining apparatus measures a pulsation rate for each time interval on the basis of biological signals detected from the subject, and determines whether the subject is trying to be awake from a change in the pulsation rate, and corrects the subject's arousal level when the subject is trying to be awake.

### CROSS-REFERENCE TO RELATED APPLICATION(S)

This application is based upon and claims the benefit of priority of the prior Japanese Patent Application No. 2014-161920, filed on Aug. 7, 2014, the entire contents of which are incorporated herein by reference.

### FIELD

The embodiment discussed herein is directed to an arousal-level determining program and the like.

### BACKGROUND

Although the total number of traffic accidents is decreasing, accidents caused by human error have not so decreased. One of causes of the accidents caused by human error is driver's drowsiness while driving. Therefore, there is a need for developing a technology to issue a warning to a driver on the basis of the level of arousal while driving, thereby preventing the driver from causing an accident.

There are various technologies for measuring the level of arousal. For example, there is a technology to extract an area surrounding driver's eye, including the upper and lower eyelids, from a face area in a photographed image of a driver and calculate a distance between the highest point of the upper eyelid and the lowest point of the lower eyelid on the basis of a difference in luminance between the eyeball and the eyelid, thereby finding the level of arousal. Furthermore, there is a technology to acquire subject's pulse signals and determine the level of arousal on the basis of changes in pulse-interval fluctuation frequency.

Patent Literature 1: Japanese Laid-open Patent Publication No. 2009-279099

Patent Literature 2: Japanese Laid-open Patent Publication No. 2012-093867

Patent Literature 3: Japanese Laid-open Patent Publication No. 2012-104068

Patent Literature 4: Japanese Laid-open Patent Publication No. 2012-234398

Patent Literature 5: Japanese Patent No. 5189893 Patent Literature 6: Japanese Laid-open Patent Publication No. 2013-252764

Patent Literature 7: Japanese Patent No. 5447335

However, the above-mentioned conventional technologies have a problem that they fail to suppress the decrease in accuracy of determination of one's drowsiness while trying to be awake.

While trying to be awake means a state in which one is feeling drowsy, though is struggling to keep awake against his/her drowsiness. For example, while a driver is trying to be awake, the movement of his/her eyelids and the pulse-interval fluctuation frequency are different from those in a normal drowsy state. Therefore, an arousal level found on the basis of the movement of the eyelids or the pulse-interval fluctuation frequency like the conventional technologies may sometimes be different from an actual arousal level.

### SUMMARY

According to an aspect of an embodiment, a computer-readable recording medium has stored therein an arousal-level determining program that causes a computer to execute a process including first determining a subject's arousal level on the basis of a biological signal detected from a subject; measuring a pulsation rate for each time interval on the basis of biological signals detected from the subject; second determining whether the subject is trying to be awake from a change in the pulsation rate; and correcting the subject's arousal level when the subject is trying to be awake.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a functional block diagram illustrating a configuration of an arousal-level determining apparatus according to a present embodiment;

FIG. 2 is a diagram illustrating an example of heartbeat signal data;

FIG. 3 is a diagram illustrating an example of heartbeat-interval variation data;

FIG. 4 is a diagram illustrating a relationship between frequency and spectral density;

FIG. 5A is a diagram for explaining a process of determining a drowsiness level;

FIG. 5B is a box plot illustrating the greatest and smallest values of heart rates in a napping state, while trying to be awake, and in an arousal state;

FIG. 6 is a diagram illustrating a relationship between drowsiness level and while trying to be awake;

FIG. 7 is a diagram illustrating an example of the data structure of a parameters table;

FIG. 8 is a flowchart illustrating a processing procedure of the arousal-level determining apparatus according to the present embodiment;

FIG. 9 is a flowchart illustrating a procedure of a drowsiness-level determining process;

FIG. 10 is a flowchart illustrating a procedure of a while-trying-to-be-awake determining process;

FIG. 11 is a flowchart illustrating a procedure of a process of setting parameters; and

FIG. 12 is a diagram illustrating an example of a computer that executes an arousal-level determining program.

### DESCRIPTION OF EMBODIMENTS

Preferred embodiments of the present invention will be explained with reference to accompanying drawings. Incidentally, this invention is not limited to the embodiment described below.

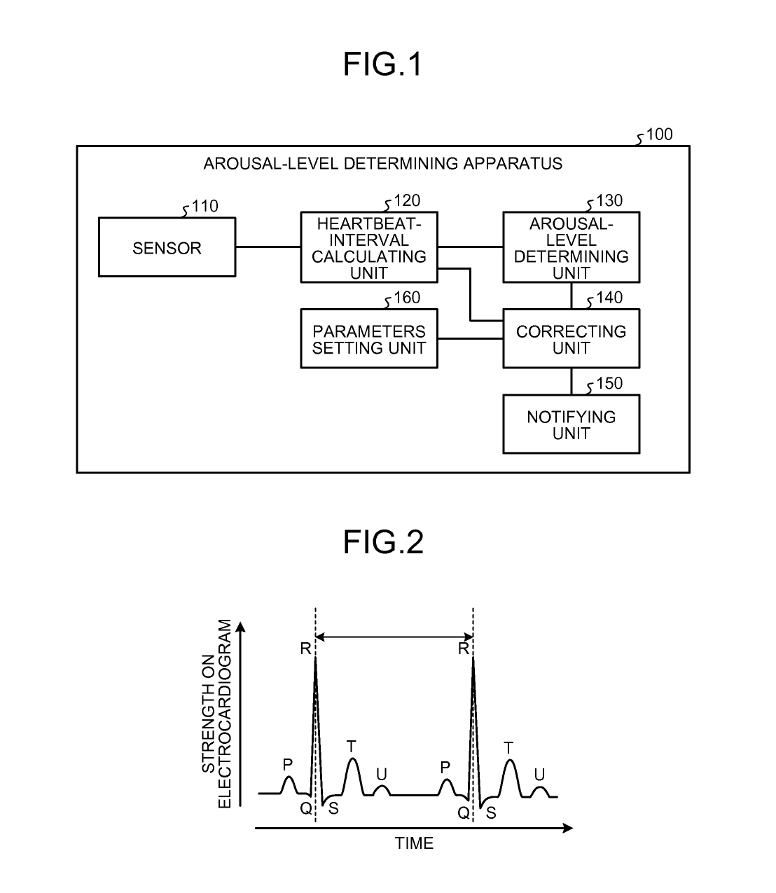

An example of a configuration of the arousal-level determining apparatus according to the present embodiment is explained. FIG. 1 is a functional block diagram illustrating the configuration of the arousal-level determining apparatus according to the present embodiment. As illustrated in FIG. 1, this arousal-level determining apparatus 100 includes a sensor 110, a heartbeat-interval calculating unit 120, an arousal-level determining unit 130, a correcting unit 140, a notifying unit 150, and a parameters setting unit 160.

Although not illustrated in FIG. 1, the arousal-level determining apparatus shall be assumed to have been installed, for example, in a vehicle driven by a subject.

The sensor 110 is a sensor for detecting a subject's heartbeat signal. The sensor 110 shall be assumed to have been installed on a steering wheel of the vehicle. In the present embodiment, there is explained an example where the sensor 110 detects a heartbeat signal; alternatively, the sensor 110 can detect a subject's pulse signal. The sensor 110 outputs data of the heartbeat signal to the heartbeat-interval calculating unit 120. Hereinafter, data of a heartbeat signal is referred to as heartbeat signal data.

FIG. 2 is a diagram illustrating an example of heartbeat signal data. As illustrated in FIG. 2, heartbeat signal data has a waveform composed of waves called P, Q, R, S, and T waves.

The heartbeat-interval calculating unit 120 is a processing unit that detects an amplitude peak of a heartbeat signal on the basis of heartbeat signal data and detects a time interval between detected amplitude peaks of heartbeat signals. The time interval between detected amplitude peaks of heartbeat signals is referred to as a heartbeat interval. With FIG. 2, processing by the heartbeat-interval calculating unit 120 is explained. As illustrated in FIG. 2, the heartbeat-interval calculating unit 120 detects a point R at which the amplitude of a heartbeat signal is equal to or greater than a threshold, i.e., an amplitude peak, and detects an interval between detected points R as an amplitude interval. The heartbeat-interval calculating unit 120 outputs data of the detected heartbeat interval to the arousal-level determining unit 130 and the correcting unit 140. Hereinafter, data of a heartbeat interval is referred to as heartbeat interval data.

The arousal-level determining unit 130 is a processing unit that determines a subject's drowsiness level on the basis of heartbeat interval data. For example, the arousal-level determining unit 130 calculates spectral density corresponding to a heartbeat, and determines a drowsiness level on the basis of a local maximum value of spectral density and a frequency corresponding to the local maximum value of spectral density. The arousal-level determining unit 130 outputs a result of the determination of the drowsiness level to the correcting unit 140.

There is explained an example of how the arousal-level determining unit 130 calculates spectral density corresponding to a heartbeat. The arousal-level determining unit 130 generates data of heartbeat intervals which vary with time on the basis of heartbeat interval data. Hereinafter, data of heartbeat intervals which vary with time is referred to as heartbeat-interval variation data.

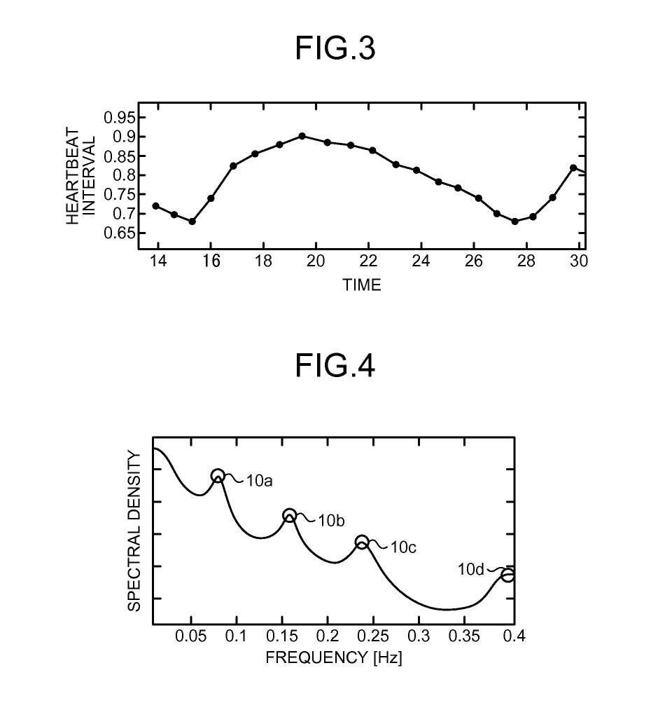

FIG. 3 is a diagram illustrating an example of heartbeat-interval variation data. In FIG. 3, the vertical axis is an axis indicating a heartbeat interval, and the horizontal axis is an axis indicating time. As illustrated in FIG. 3, a heartbeat interval varies with time.

The arousal-level determining unit 130 calculates a relationship between frequency and spectral density on the basis of the heartbeat-interval variation data. FIG. 4 is a diagram illustrating the relationship between frequency and spectral density. In FIG. 4, the vertical axis is an axis indicating spectral density, and the horizontal axis is an axis indicating frequency. In an example illustrated in FIG. 4, the spectral density reaches a local maximum at points 10a, 10b, 10c, and 10d. Hereinafter, data indicating the relationship between spectral density and frequency is referred to as spectral density data.

Here, the arousal-level determining unit 130 can use any method to calculate the relationship between spectral density and frequency, but can calculate spectral density by using an autoregressive (AR) model. As disclosed in Non-patent Literature (Sato Shunsuke, Kikkawa Sho, and Kiryu Toru, “Introduction to biosignal processing”, CORONA PUBLISHING CO., LTD., 2004), an AR model is a model that expresses previous time-series data at a certain point in linear combination, and is characterized by being able to obtain distinct local maximum points even from a small number of data as compared with Fourier transform. Incidentally, the arousal-level determining unit 130 can calculate the relationship between spectral density and frequency by using Fourier transform.

A time-series x(s), p-th order AR model can be represented by the following equation (1a) using an AR parameter a(m), which is a weight to a previous value, and an error term e(s). Ideally, e(s) in equation (1a) is white noise.

$$x ( s ) = {\sum }_{m = 1}^{P} a ( m ) x ( s - m ) + e ( s ) \qquad ( 1 a )$$

Spectral density_{AR}(f) is represented by the following equation (2a), where p denotes an identification order, fₛ denotes a sampling frequency, εₚ denotes an identification error, and â_{P}(k) denotes a k-th AR parameter. The arousal-level determining unit 130 calculates spectral density data on the basis of equation (2a) and the heartbeat-interval variation data.

$${P}_{AR} ( f ) = \frac{1}{{f}_{S}} \frac{{ɛ}_{P}}{{\vert 1 + {\sum }_{k = 1}^{P} {\hat{a}}_{P} ( k ) e \frac{- 2 π j kf}{{f}_{k}} \vert}^{2}} \qquad ( 2 a )$$

Subsequently, there is explained an example of how the arousal-level determining unit 130 determines a drowsiness level on the basis of a local maximum value of spectral density and a frequency corresponding to the local maximum value of spectral density. Hereinafter, a local maximum value of spectral density is referred to as maximum spectral density. Furthermore, a frequency corresponding to maximum spectral density is referred to as a maximum frequency.

The arousal-level determining unit 130 calculates a frequency f that satisfies a relation represented by the following equation (3a) as a maximum frequency. The arousal-level determining unit 130 substitutes the maximum frequency into equation (2a), thereby obtaining maximum spectral density.

$$\frac{d {P}_{AR} ( f )}{d f} = 0 \qquad ( 3 a )$$

The arousal-level determining unit 130 selects any of the maximum spectral densities on the basis of the spectral density data. For example, the arousal-level determining unit 130 selects any of the maximum spectral densities 10a to 10d illustrated in FIG. 4, and focuses on the selected maximum spectral density and a temporal change in a maximum frequency corresponding to the selected maximum spectral density.

For example, the arousal-level determining unit 130 plots a relationship between maximum spectral density to be focused on and a maximum frequency corresponding to the maximum spectral density on a graph. A point on the graph set by maximum spectral density and its corresponding maximum frequency is referred to as a feature point. The arousal-level determining unit 130 determines a subject's drowsiness level on the basis of the position of a feature point on the graph.

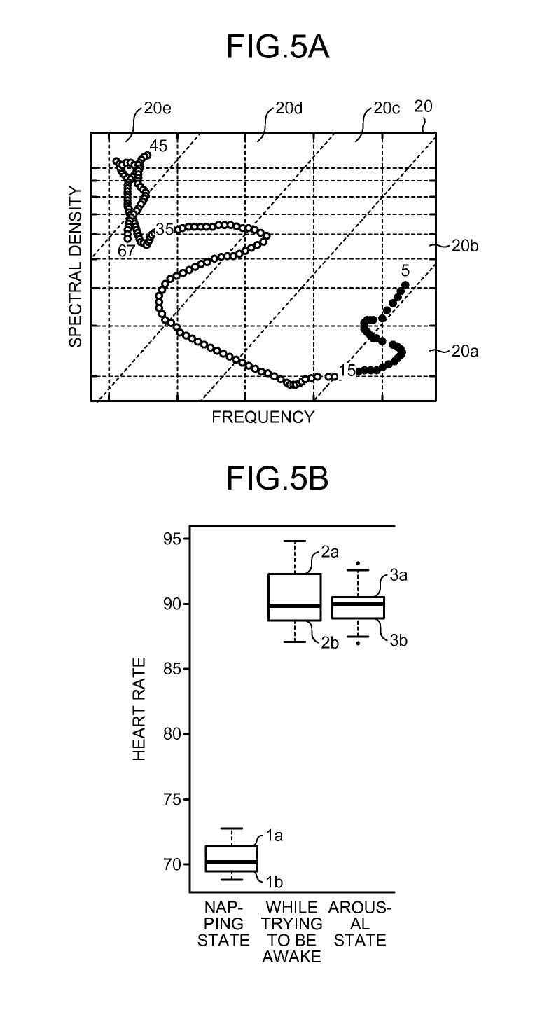

FIGS. 5A and 5B are diagrams for explaining a process of determining a drowsiness level. The vertical axis of a graph 20 illustrated in FIGS. 5A and 5B is an axis corresponding to maximum spectral density, and the horizontal axis is an axis corresponding to maximum frequency. Points plotted on the graph 20 indicate a locus of feature points. For example, the lower the maximum frequency and the higher the maximum spectral density, the higher the subject's drowsiness level gets.

For example, when the position of a feature point is included in an area 20a, the arousal-level determining unit 130 determines that a subject's drowsiness level is “drowsiness level 1”. When the position of a feature point is included in an area 20b, the arousal-level determining unit 130 determines that a subject's drowsiness level is “drowsiness level 2”. When the position of a feature point is included in an area 20c, the arousal-level determining unit 130 determines that a subject's drowsiness level is “drowsiness level 3”. When the position of a feature point is included in an area 20d, the arousal-level determining unit 130 determines that a subject's drowsiness level is “drowsiness level 4”. When the position of a feature point is included in an area 20e, the arousal-level determining unit 130 determines that a subject's drowsiness level is “drowsiness level 5”.

To return to the explanation of FIG. 1, the correcting unit 140 measures a heart rate for each time interval on the basis of heartbeat interval data, and determines whether a subject is trying to be awake from a change in the heart rate. When the subject is trying to be awake, the correcting unit 140 corrects a result of determination by the arousal-level determining unit 130. The correcting unit 140 outputs information on the corrected subject's drowsiness level to the notifying unit 150. On the other hand, when the subject is not trying to be awake, the correcting unit 140 outputs a result of determination by the arousal-level determining unit 130 as is to the notifying unit 150.

There is explained an example of how the correcting unit 140 determines whether a subject is trying to be awake. The correcting unit 140 measures a heart rate by comparing a window having a predetermined time width with heartbeat interval data. For example, the correcting unit 140 calculates an average value of heartbeat intervals included in the window, and finds a reciprocal of the average value, thereby calculating a heart rate. When a heart rate per minute is to be calculated, the correcting unit 140 just has to multiply the reciprocal of the average value by 60. The correcting unit 140 repeatedly performs the above-described process, moving the position of the window, thereby measuring a heart rate for each time interval.

After having calculated the heart rate for each time interval, the correcting unit 140 sorts the calculated heart rates in descending order. The correcting unit 140 excludes, out of the sorted heart rates, some lowest-ranked heart rates and some highest-ranked heart rates as outliers on the basis of an outlier threshold. For example, when the outlier threshold is 25%, the correcting unit 140 identifies the number of heart rates corresponding to 25% out of the number of all the heart rates, and excludes an identified number of heart rates from the highest and lowest-ranked heart rates in the sorted heart rates. For example, when there are 100 heart rates, the first to twenty-fifth heart rates and the seventy-fifth to hundredth heart rates in the sorted heart rates are excluded as outliers.

The correcting unit 140 detects the greatest value of heart rate and the smallest value of heart rate out of the heart rates excluding the outliers. The correcting unit 140 calculates a variance value by subtracting the smallest value of heart rate from the greatest value of heart rate. When the variance value is equal to or greater than a threshold, the correcting unit 140 determines that the subject is trying to be awake.

FIG. 5B is a box plot illustrating the greatest and smallest values of heart rates in a napping state, while trying to be awake, and in an arousal state. As illustrated in FIG. 5B, in the napping state, the greatest value of heart rate is la, and the smallest value is 1b. While trying to be awake, the greatest value of heart rate is 2a, and the smallest value is 2b. In the arousal state, the greatest value of heart rate is 3a, and the smallest value is 3b. As illustrated in FIG. 5B, a difference in between the greatest value 2a and smallest value 2b of heart rate while trying to be awake is larger than a difference in between the greatest value 1a and smallest value 1b of heart rate in the napping state and a difference in between the greatest value 3a and smallest value 3b of heart rate in the arousal state.

There is explained a process performed by the correcting unit 140 when having determined that a subject is trying to be awake. For example, when a subject is trying to be awake, the correcting unit 140 corrects a drowsiness level acquired from the arousal-level determining unit 130 to drowsiness level 4 or drowsiness level 5. Either drowsiness level 4 or drowsiness level 5 the drowsiness level while trying to be awake is to be is set by an administrator in advance.

When a subject is trying to be awake, the subject is very drowsy, though is struggling against his/her drowsiness; therefore, the arousal-level determining unit 130 does not determine the subject's drowsiness level properly.

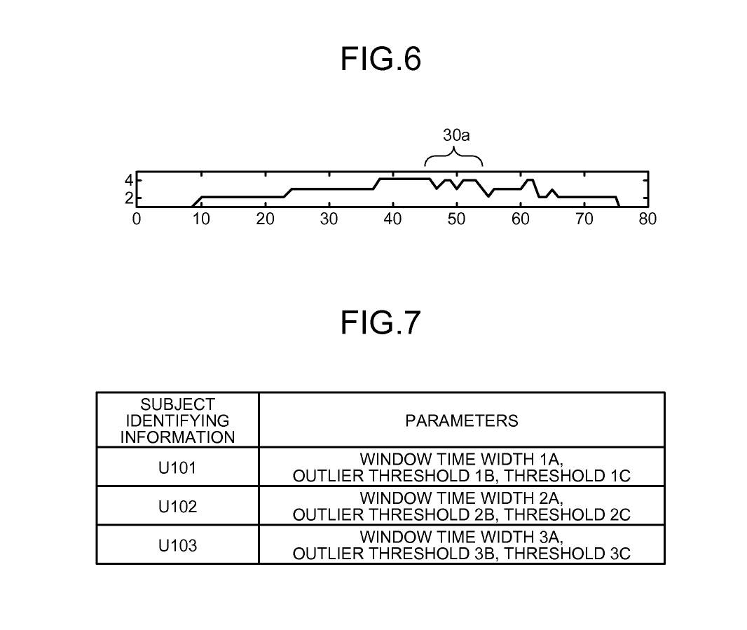

FIG. 6 is a diagram illustrating a relationship between drowsiness level and while trying to be awake. The vertical axis in FIG. 6 corresponds to drowsiness level, and the horizontal axis corresponds to time. For example, when it shall be assumed that a user is trying to be awake during a time period 30a, a user's drowsiness level is supposed to be high during the time period 30a; however, the drowsiness level may sometimes be low. Therefore, the correcting unit 140 corrects the drowsiness level during the time period 30a to drowsiness level 4 or 5.

The notifying unit 150 is a processing unit that issues a warning to a subject on the basis of a drowsiness level. For example, when a drowsiness level has reached drowsiness level 4 or higher, the notifying unit 150 issues warning. The notifying unit 150 can issue an audio warning, or can display an image of a warning on a display installed in a vehicle.

The parameters setting unit 160 is a processing unit that outputs subject's parameters to the correcting unit 140. The subject's parameters include a window time width, an outlier threshold, and a threshold. Out of these, the window time width is information used when the correcting unit 140 calculates a heart rate. The outlier threshold is information used when the correcting unit 140 excludes outliers. The threshold is information corresponding to a threshold that the correcting unit 140 compares with a variance value. The correcting unit 140 performs the process of determining whether a subject is trying to be awake on the basis of parameters which are set by the parameters setting unit 160.

The parameters setting unit 160 sets parameters in the correcting unit 140 by using a parameters table. FIG. 7 is a diagram illustrating an example of the data structure of the parameters table. As illustrated in FIG. 7, the parameters table associates subject identifying information with parameters. The subject identifying information is information for uniquely identifying a subject. The parameters correspond to the above-described window time width, outlier threshold, and threshold.

For example, a subject inputs subject identifying information to the parameters setting unit 160 by operating an input device (not illustrated). The parameters setting unit 160 acquires the subject identifying information from the input device, and identifies parameters corresponding to the subject by comparing the subject identifying information with the parameters table, and then sets the identified parameters in the correcting unit 140.

Incidentally, the parameters setting unit 160 can be configured to optimize parameters stored in the parameters table by performing the following process. The parameters setting unit 160 defines sensitivity and specificity, and searches for parameters resulting in the maximum value of sensitivity and the maximum value of specificity with respect to each subject, and then updates the parameters table on the basis of a result of the search. Sensitivity is defined by the following equation (1). Specificity is defined by the following equation (2).

Sensitivity=(The number of determinations that a subject was trying to be awake)/(the true total number of times the subject was trying to be awake) (1)

Specificity=(The number of determinations that a subject was not trying to be awake)/(the true total number of times the subject was not trying to be awake) (2)

In equation (1), the number of determinations that a subject was trying to be awake means the number of times the correcting unit 140 determined that the subject was trying to be awake in a first predetermined period by the currently-set parameters.

In equation (1), the true total number of times the subject was trying to be awake is identified on the basis of photographed images of the subject's face. The parameters setting unit 160 acquires images from a camera (not illustrated), and measures the number of times the subject blinked. For example, when the number of times the subject blinked in a second predetermined period is equal to or more than the predetermined number of times, the parameters setting unit 160 determines that the subject is trying to be awake. The second predetermined period shall be shorter than the first predetermined period. The parameters setting unit 160 sets the number of determinations that the subject was trying to be awake in the first predetermined period as the true total number of times the subject was trying to be awake. The first and second predetermined periods are set properly by an administrator.

In equation (2), the number of determinations that a subject was not trying to be awake means the number of times the correcting unit 140 determined that the subject was not trying to be awake in the first predetermined period by the currently-set parameters.

In equation (2), the true total number of times the subject was not trying to be awake is identified on the basis of photographed images of the subject's face. The parameters setting unit 160 acquires images from the camera (not illustrated), and measures the number of times the subject blinked. For example, when the number of times the subject blinked in the second predetermined period is less than the predetermined number of times, the parameters setting unit 160 determines that the subject is not trying to be awake. The parameters setting unit 160 sets the number of determinations that the subject was not trying to be awake in the first predetermined period as the true total number of times the subject was not trying to be awake.

The parameters setting unit 160 prepares several different types of parameters for each subject, and calculates sensitivity and specificity, changing the parameters set in the correcting unit 140. The parameters setting unit 160 sets parameters resulting in high sensitivity and high specificity with respect to each subject. For example, the parameters setting unit 160 searches for parameters resulting in higher sensitivity and higher specificity than their respective predetermined thresholds.

Incidentally, the parameters setting unit 160 can calculate sensitivity by using the following equation (3) instead of equation (1), and can calculate specificity by using the following equation (4) instead of equation (2).

Sensitivity=(The number of determinations that a subject was trying to be awake)/(the true total number of times it failed to determine that the subject was drowsy in spite of the fact that the subject was drowsy) (3)

Specificity=(The number of determinations that a subject was not trying to be awake)/(the true total number of times it failed to determine that the subject was drowsy in spite of the fact that the subject was drowsy) (4)

The true total number of times it failed to determine that the subject was drowsy in spite of the fact that the subject was drowsy in equations (3) and (4) is the number identified on the basis of photographed images of the subject's face and results of determination by the arousal-level determining unit 130. For example, when the number of times the subject blinked in the second predetermined period is equal to or more than the predetermined number of times, the parameters setting unit 160 that the subject is in a drowsy state. Then, when the drowsiness level output to the notifying unit 150 by the correcting unit 140 in the second predetermined period is lower than drowsiness level 4, the true total number of times it failed to determine that the subject was drowsy in spite of the fact that the subject was drowsy is incremented by one. The parameters setting unit 160 measures the true total number of times it failed to determine that the subject was drowsy in spite of the fact that the subject was drowsy in the first predetermined period.

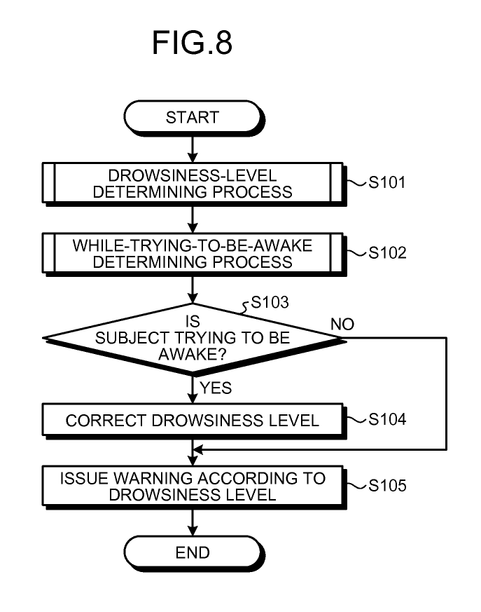

Subsequently, there is explained a processing procedure of the arousal-level determining apparatus 100 according to the present embodiment. FIG. 8 is a flowchart illustrating the processing procedure of the arousal-level determining apparatus according to the present embodiment. As illustrated in FIG. 8, the arousal-level determining unit 130 of the arousal-level determining apparatus 100 performs a drowsiness-level determining process (Step S101).

The correcting unit 140 of the arousal-level determining apparatus 100 performs a while-trying-to-be-awake determining process (Step S102). The correcting unit 140 determines whether a subject is trying to be awake (Step S103). When the subject is not trying to be awake (NO at Step S103), the processing proceeds to Step S105.

On the other hand, when the subject is trying to be awake (YES at Step S103), the correcting unit 140 corrects the drowsiness level (Step S104). The notifying unit 150 of the arousal-level determining apparatus 100 issues a warning according to the drowsiness level (Step S105).

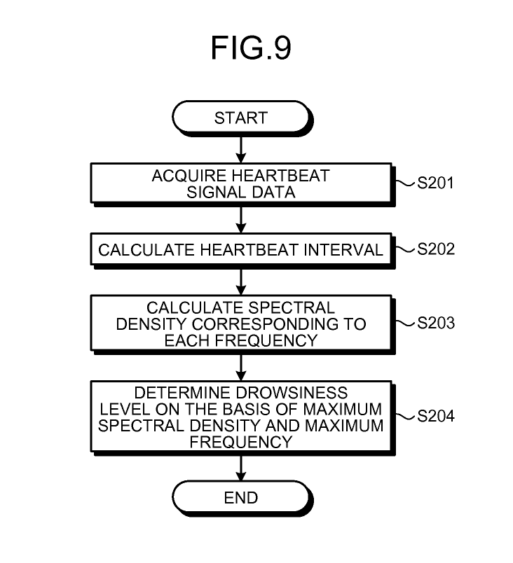

Subsequently, there is explained a procedure of the drowsiness-level determining process illustrated at Step S101 in FIG. 8. FIG. 9 is a flowchart illustrating the procedure of the drowsiness-level determining process. As illustrated in FIG. 9, the heartbeat-interval calculating unit 120 of the arousal-level determining apparatus 100 acquires heartbeat signal data from the sensor 110 (Step S201).

The heartbeat-interval calculating unit 120 calculates a heartbeat interval (Step S202). The arousal-level determining unit 130 of the arousal-level determining apparatus 100 calculates spectral density corresponding to each frequency (Step S203). The arousal-level determining unit 130 determines a drowsiness level on the basis of maximum spectral density and a maximum frequency (Step S204).

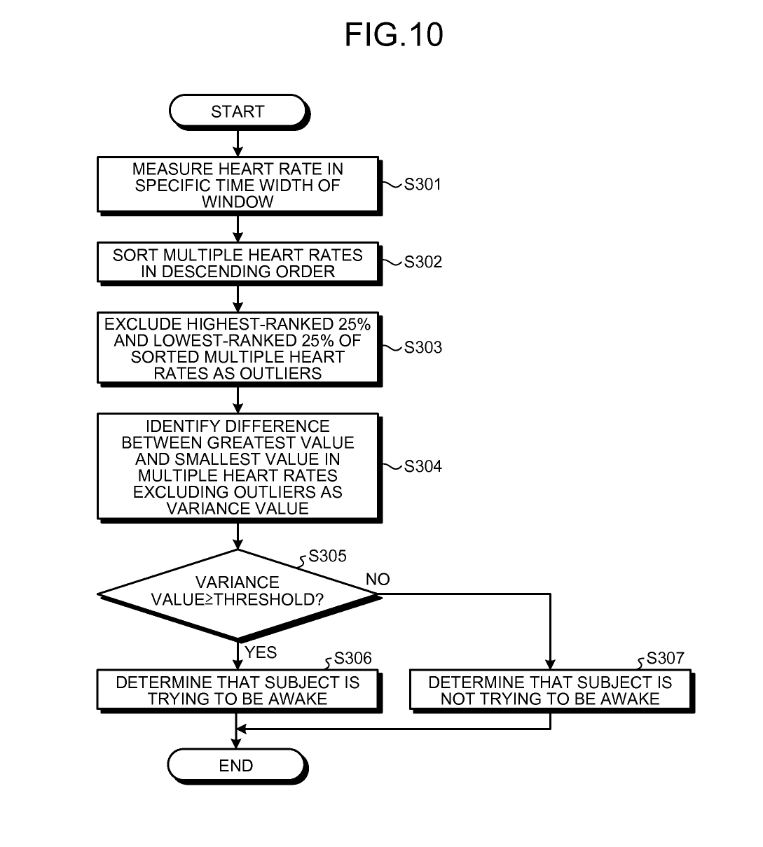

Subsequently, there is explained a procedure of the while-trying-to-be-awake determining process illustrated at Step S102 in FIG. 8. FIG. 10 is a flowchart illustrating the procedure of the while-trying-to-be-awake determining process. As illustrated in FIG. 10, the correcting unit 140 of the arousal-level determining apparatus 100 measures a heart rate in a specific time width of window (Step S301).

The correcting unit 140 sorts multiple heart rates in descending order (Step S302). The correcting unit 140 excludes the highest-ranked 25% and lowest-ranked 25% of the sorted multiple heart rates as outliers (Step S303).

The correcting unit 140 identifies a difference between the greatest value and the smallest value in multiple heart rates excluding the outliers as a variance value (Step S304). The correcting unit 140 determines whether the variance value is equal to or greater than a threshold (Step S305). When the variance value is equal to or greater than the threshold (YES at Step S305), the correcting unit 140 determines that a subject is trying to be awake (Step S306). On the other hand, when the variance value is smaller than the threshold (NO at Step S305), the correcting unit 140 determines that a subject is not trying to be awake (Step S307).

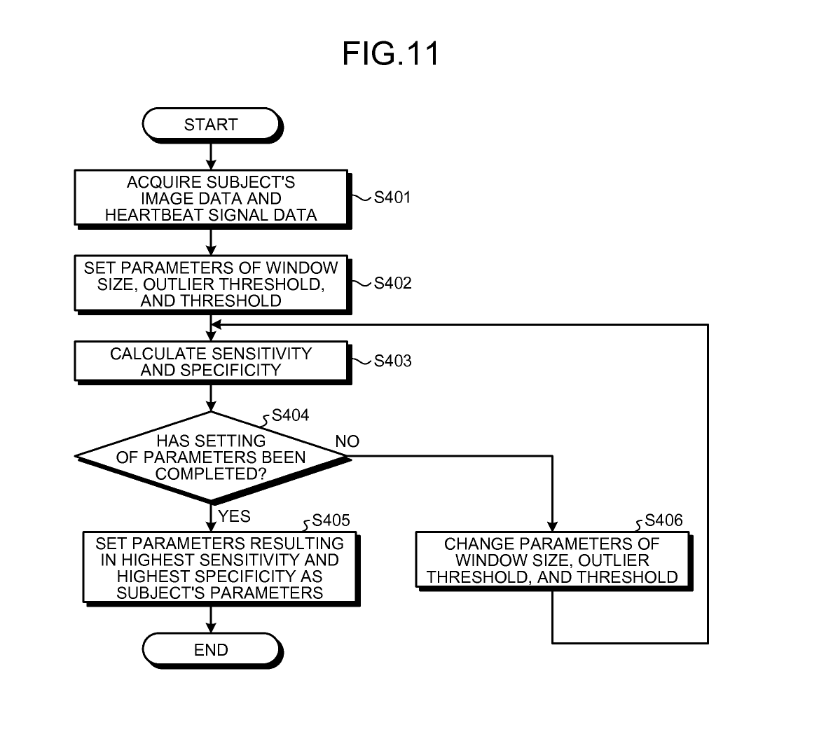

Subsequently, there is explained an example of how to identify subject's parameters. FIG. 11 is a flowchart illustrating a procedure of a process of setting parameters. The arousal-level determining apparatus 100 performs the process illustrated in FIG. 11 with respect to each subject.

As illustrated in FIG. 11, the arousal-level determining apparatus 100 acquires subject's image data and heartbeat signal data (Step S401). The parameters setting unit 160 of the arousal-level determining apparatus 100 sets a window size, an outlier threshold, and a threshold in the correcting unit 140 (Step S402).

The parameters setting unit 160 calculates sensitivity and specificity (Step S403). The parameters setting unit 160 determines whether the setting of parameters has been completed (Step S404). When the setting of parameters has been completed (YES at Step S404), the parameters setting unit 160 sets parameters resulting in the highest sensitivity and the highest specificity as subject's parameters (Step S405).

On the other hand, when the setting of parameters has not been completed (NO at Step S404), the parameters setting unit 160 changes the parameters of the window size, the outlier threshold, and the threshold set in the correcting unit 140 (Step S406), and proceeds to Step S403.

Subsequently, advantageous effects of the arousal-level determining apparatus 100 according to the present embodiment are explained. The arousal-level determining apparatus 100 measures a heart rate for each time interval on the basis of heartbeat signal data, and determines whether a subject is trying to be awake from a change in the heart rate, and, when the subject is trying to be awake, corrects a subject's drowsiness level. Therefore, the arousal-level determining apparatus 100 can suppress the decrease in accuracy of determination of subject's drowsiness while trying to be awake. For example, while the subject is trying to be awake, changes in subject's autonomic nerve activity are substantial, and the accuracy of subject's drowsiness level decreases; however, the decrease in the accuracy of subject's drowsiness level while trying to be awake can be addressed by correcting the drowsiness level on the basis of whether the subject is trying to be awake.

Furthermore, the arousal-level determining apparatus 100 excludes, out of multiple heart rates, some highest-ranked heart rates and some lowest-ranked heart rates as outliers from the multiple heart rates, and determines whether the subject is trying to be awake on the basis of a variance value of multiple heart rates excluding the outliers. Therefore, it is possible to accurately determine whether the subject is trying to be awake.

Moreover, the arousal-level determining apparatus 100 sets a threshold for each subject, and, when a variance value of multiple heart rates is equal to or greater than a threshold corresponding to a subject, determines that the subject is trying to be awake. Therefore, it is possible to determine whether the subject is trying to be awake according to subject-specific features.

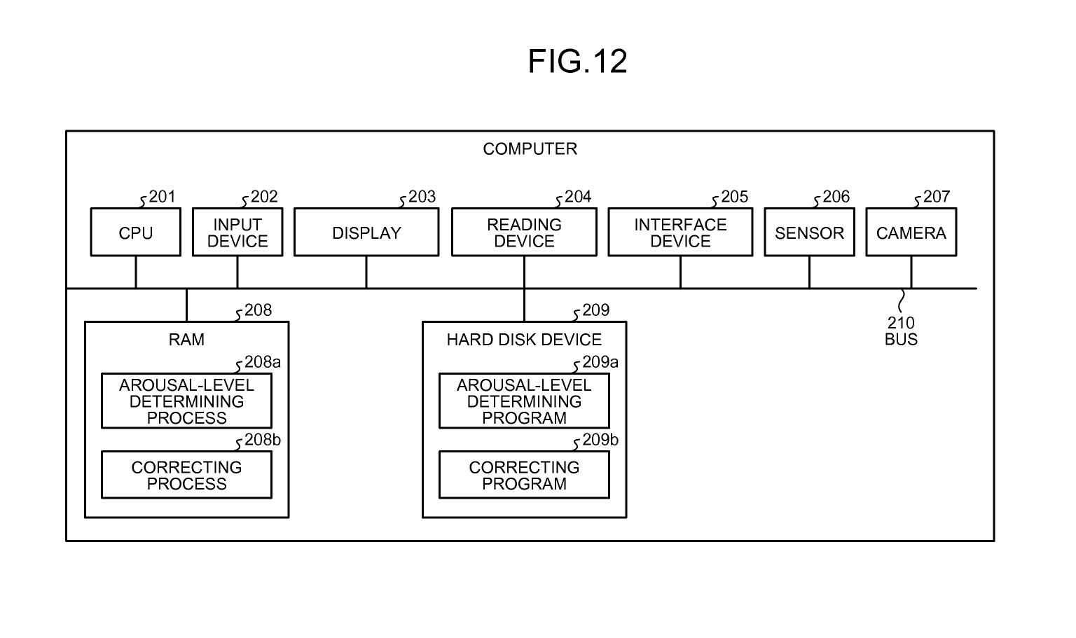

Subsequently, there is explained an example of a computer that executes an arousal-level determining program realizing the same functions as the arousal-level determining apparatus 100 described in the above embodiment. FIG. 12 is a diagram illustrating an example of the computer that executes the arousal-level determining program.

As illustrated in FIG. 12, a computer 200 includes a CPU 201 that executes a variety of arithmetic processing, an input device 202 that receives input of data from a user, and a display 203. Furthermore, the computer 200 includes a reading device 204 that reads a program etc. from a storage medium and an interface device 205 that transfers data to and from another computer via a network. Moreover, the computer 200 includes a sensor 206 and a camera 207. Furthermore, the computer 200 includes a RAM 208 for temporarily storing therein a variety of information and a hard disk device 209. These devices 201 to 209 are connected to a bus 210.

The hard disk device 209 reads out an arousal-level determining program 209a and a correcting program 209b, and expands the read programs into the RAM 208. The arousal-level determining program 209a serves as an arousal-level determining process 208a. The correcting program 209b serves as a correcting process 208b. For example, the arousal-level determining process 208a corresponds to the arousal-level determining unit 130. The correcting process 208b corresponds to the correcting unit 140.

Incidentally, the arousal-level determining program 209a and the correcting program 209b do not always have to be stored in the hard disk device 209 from the beginning. For example, these programs can be stored in a “portable physical medium”, such as a flexible disk (FD), a CD-ROM, a DVD, a magnet-optical disk, or an IC card, to be inserted into the computer 200. Then, the computer 200 can read out and execute the arousal-level determining program 209a and the correcting program 209b.

According to an embodiment of the present invention, it is possible to suppress the decrease in accuracy of determination of one's drowsiness while trying to be awake.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. A non-transitory computer-readable recording medium having stored therein an arousal-level determining program that causes a computer to execute a process comprising:
first determining a subject's arousal level on the basis of a biological signal detected from a subject;
measuring a pulsation rate for each time interval on the basis of biological signals detected from the subject;
second determining whether the subject is trying to be awake from a change in the pulsation rate; and
correcting the subject's arousal level when the subject is trying to be awake.

2. The non-transitory computer-readable recording medium according to claim 1, wherein
the second determining excludes a predetermined number of pulsation rates from highest-ranked pulsation rate and a lowest-ranked pulsation rate as outliers out of multiple pulsation rates and determines whether the subject is trying to be awake on the basis of a variance value of multiple pulsation rates excluding the outliers.

3. The non-transitory computer-readable recording medium according to claim 2, wherein
the second determining sets a threshold for each subject and determines that the subject is trying to be awake, when a variance value of multiple pulsation rates is equal to or greater than a threshold corresponding to a subject.

4. The non-transitory computer-readable recording medium according to claim 3, wherein
the second determining sets an outlier threshold for each subject and determines a variance value of pulsation rates, out of multiple pulsation rates, the pulsation rates being excluded outliers corresponding to a subject on the basis of an outlier threshold for the subject.

5. An arousal-level determining apparatus comprising:
a processor that executes a process comprising:
first determining a subject's arousal level on the basis of a biological signal detected from a subject;
measuring a pulsation rate for each time interval on the basis of biological signals detected from the subject;
second determining whether the subject is trying to be awake from a change in the pulsation rate; and
correcting the subject's arousal level when the subject is trying to be awake.

6. The arousal-level determining apparatus according to claim 5, wherein
the second determining excludes a predetermined number of pulsation rates from highest-ranked pulsation rate and a lowest-ranked pulsation rate as outliers out of multiple pulsation rates and determines whether the subject is trying to be awake on the basis of a variance value of multiple pulsation rates excluding the outliers.

7. The arousal-level determining apparatus according to claim 6, wherein
the second determining sets a threshold for each subject and determines that the subject is trying to be awake, when a variance value of multiple pulsation rates is equal to or greater than a threshold corresponding to a subject.

8. The arousal-level determining apparatus according to claim 7, wherein
the second determining sets an outlier threshold for each subject and determines a variance value of pulsation rates, out of multiple pulsation rates, the pulsation rates being excluded outliers corresponding to a subject on the basis of an outlier threshold for the subject.
